(19) United States
(12) Patent Application Publication
(10) Pub. No.: US 2004/0142356 A1
(43) Pub. Date: Jul. 22, 2004

(54) Modified luciferase nucleic acids and methods of use

(76) Inventors: Stacey Patterson, Tampa, FL (US); Rakesh Gupta, New Delhi (IN); Gary Sayler, Blaine, TN (US); Hebe Dionisi, Chubut (AR)

Correspondence Address: Stanley A. Kim, Ph.D., Esq., Akerman Senterfitt, Suite 400, 222 Lakeview Avenue, West Palm Beach, FL, 33402-3188, US

(21) Appl. No.: 10/697,419

(22) Filed: Oct. 30, 2003

Related U.S. Application Data
(60) Provisional application No. 60/422,467, filed on Oct. 30, 2002.

(51) Int. Cl. C12Q 1/68; C12Q 1/66; C07H 21/04; C12N 9/06

(52) U.S. Cl. 435/6; 435/8; 435/191; 435/252.3; 435/320.1; 536/23.2

(57) ABSTRACT

The luxA and luxB genes from P. luminescens which encode for the luciferase protein of the bacterial luciferase system were modified to generate codon-optimized versions that are optimized for expression in mammalian cells. The codon-optimized bacterial luciferase enzyme system genes of the invention can be used to develop a mammalian bioluminescence bioreporter useful in various medical research and diagnostics applications.

### CROSS-REFERENCE TO RELATED APPLICATIONS

The present application claims the priority of U.S. provisional patent application No. 60/422,467 filed on Oct. 30, 2002.

### STATEMENT AS TO FEDERALLY SPONSORED RESEARCH

This invention was made in part with U.S. government support under grant number R21RR14169-02 awarded by the National Institutes of Health. The U.S. government may have certain rights in the invention.

### FIELD OF THE INVENTION

The invention relates generally to the fields of molecular biology, microbiology, and biosensors. More specifically, the invention relates to the development of codon-optimized luciferase system nucleotide sequences and uses thereof.

### BACKGROUND

Numerous reporter proteins have been proven to be important tools for advancing both basic and applied research. Of these, the bacterial luciferase (lux) system is particularly advantageous as it is the only bioreporter system available capable of making its own substrate and generating an autonomous signal. This property has made this bacterial bioluminescence reporter system invaluable for creating whole cell biosensors for remote sensing in prokaryotic organisms (Sayler et al., Current Opinions In Biotechnology, 12:455-460, 2001). Several lux system prokaryotic biosensors have been developed and employed in a variety of applications ranging from environmental pollutant monitoring (Sayler et al., Current Opinions In Biotechnology, 12:455-460, 2001; Ripp et al., Environmental Science and Technology, 34:846-853, 2000; Corbiser et al., Anal. Chim. Acta. 387:235-244, 1999; Kohler et al., Fresenius Journal of Analitical Chemistry, 366:769-779, 2000; King et al., Science, 249:778-781, 1990) to visualizing infections in vivo (Francis et al., Infection and Immunity, 69:3350-3358, 2001).

Despite its advantages, this technology has not previously been successfully implemented in mammalian cells due to several obstacles that prevent efficient expression of the lux proteins in mammalian cells (Naylor, L. H., Biochemical Pharmacology 58:749-757, 1999). For example, unlike polycistronic expression of multiple genes often found in bacterial systems, eukaryotic gene expression requires that each individual gene be preceded by its own promoter. This has limited the expression of the lux genes in eukaryotes to this point. Other factors that have limited expression of the lux genes in mammalian (warm-blooded) cells are protein thermo-instability and improper folding. Also, for an autonomously driven bioluminescent eukaryotic (e.g., mammalian) cell line to realize its full technological potential, the expression system must remain stable for long periods of time without the need for selective pressure. As one step in successfully implementing this technology in mammalian cells, the development of compositions and methods for efficiently expressing luciferase system components in mammalian cells is needed.

### SUMMARY

The invention relates to the development of codon-optimized nucleic acids encoding components of the bacterial luciferase system. In making the invention, the bacterial codons that naturally occur in the genes encoding LuxA and LuxB were replaced with codons optimized for expression of these genes in mammalian cells. Introducing these codon-optimized (e.g., mammalianized) sequences into mammalian cells resulted in a significant increase in LuxA and LuxB protein expression in the cells, and correspondingly a significant increase in bioluminescence in the cells. The codon-optimized nucleic acids of the invention are particularly useful for application in mammalian whole cell biosensors.

Accordingly, the invention features a nucleic acid including a codon-optimized nucleotide sequence encoding a component of a bacterial luciferase system. The codon-optimized nucleotide sequence can differ from a wild-type (WT) nucleotide sequence that encodes the component of a bacterial luciferase system by at least one of the following codon substitutions: TTT to TTC; TTA, CTA, TTG, and CTT to CTG or CTC; ATT and ATA to ATC; GTT and GTA to GTG or GTC; TCT, TCA, and TCG to TCC; CCA and CCG to CCC or CCT; ACT, ACA and ACG to ACC; GCA and GCG to GCT or GCC; TAT to TAC; CAT to CAC; CAA to CAG; AAT to AAC; AAA to AAG; GAT to GAC; GAA to GAG; TGT to TGC; CGT and CGA to CGC, CGG, and AGA; AGT to AGC; and GGT and GGA to GGC or GGG. The component of a bacterial luciferase system can be a LuxA polypeptide and the codon-optimized nucleotide sequence can be SEQ ID NO:1. The component of a bacterial luciferase system can also be a LuxB polypeptide and the codon-optimized nucleotide sequence can be SEQ ID NO:2. Nucleic acids of the invention can further include a regulatory element operably linked to the codon-optimized nucleotide sequence. The regulatory element can be an enhancer.

In another aspect, the invention features a cell having a nucleic acid including a codon-optimized nucleotide sequence that encodes a component of a bacterial luciferase system. The cell can be a mammalian cell and can be immobilized on a substrate. The codon-optimized nucleotide sequence can differ from a WT nucleotide sequence that encodes the component of a bacterial luciferase system by at least one of the following codon substitutions: TTT to TTC; TTA, CTA, TTG, and CTT to CTG or CTC; ATT and ATA to ATC; GTT and GTA to GTG or GTC; TCT, TCA, and TCG to TCC; CCA and CCG to CCC or CCT; ACT, ACA and ACG to ACC; GCA and GCG to GCT or GCC; TAT to TAC; CAT to CAC; CAA to CAG; AAT to AAC; AAA to AAG; GAT to GAC; GAA to GAG; TGT to TGC; CGT and CGA to CGC, CGG, and AGA; AGT to AGC; and GGT and GGA to GGC or GGG. The component of a bacterial luciferase system can be a LuxA polypeptide and the codon-optimized nucleotide sequence can be SEQ ID NO:1. The component of a bacterial luciferase system can also include a LuxB polypeptide and the codon-optimized nucleotide sequence can be SEQ ID NO:2. The codon-optimized nucleotide sequence can be operably linked to a regulatory element such as an enhancer.

Within the invention is a method including the step of introducing into a mammalian cell a nucleic acid including a codon-optimized nucleotide sequence encoding a component of a bacterial luciferase system. The codon-optimized nucleotide sequence can differ from a WT nucleotide sequence that encodes the component of a bacterial luciferase system by at least one of the following codon substitutions: TTT to TTC; TTA, CTA, TTG, and CTT to CTG or CTC; ATT and ATA to ATC; GTT and GTA to GTG or GTC; TCT, TCA, and TCG to TCC; CCA and CCG to CCC or CCT; ACT, ACA and ACG to ACC; GCA and GCG to GCT or GCC; TAT to TAC; CAT to CAC; CAA to CAG; AAT to AAC; AAA to AAG; GAT to GAC; GAA to GAG; TGT to TGC; CGT and CGA to CGC, CGG, and AGA; AGT to AGC; and GGT and GGA to GGC or GGG. The component of a bacterial luciferase system can be a LuxA polypeptide and the codon-optimized nucleotide sequence can be SEQ ID NO:1. The component of a bacterial luciferase system can also be a LuxB polypeptide and the codon-optimized nucleotide sequence can be SEQ ID NO:2. The codon-optimized nucleotide sequence can be operably linked to a regulatory element such as an enhancer.

Unless otherwise defined, all technical-terms used herein have the same meaning as commonly understood by one of ordinary skill in the art to which this invention belongs.

As used herein, a “nucleic acid” or a “nucleic acid molecule” means a chain of two or more nucleotides such as RNA (ribonucleic acid) and DNA (deoxyribonucleic acid).

As used herein, “protein” or “polypeptide” are used synonymously to mean any peptide-linked chain of amino acids, regardless of length or post-translational modification, e.g., glycosylation or phosphorylation.

The phrase “codon-optimized nucleotide sequence” means one that differs from a naturally occurring sequence by at least one (e.g., 2, 3, 4, 5, 10, 25, 50, 100, 200 or more or all) codon substitution, the codon substitution being one that promotes a higher level of expression of the nucleic acid in a given cell, than does the naturally occurring sequence.

Examples of codons that are more preferred for expression in mammalian cells include: GCC encoding alanine, TGC encoding cysteine, GAC encoding aspartic acid, GAG encoding glutamic acid, TTC encoding phenylalanine, GGC encoding glycine, CAC encoding histidine, ATC encoding isoleucine, AAG encoding lysine, CTG encoding leucine, AAC encoding asparagine, CCC encoding proline, CAG encoding glutamine, CGC encoding arginine, AGC encoding serine, ACC encoding threonine, GTG encoding valine, and TAC encoding tyrosine.

By the phrase “a component of a bacterial luciferase system” is meant LuxA, LuxB, LuxC, LuxD, LuxE, or FMN oxidoreductase.

As used herein, the term “vector” refers to a nucleic acid molecule capable of transporting another nucleic acid to which it has been linked. Vectors capable of directing the expression of genes to which they are operatively linked are referred to herein as “expression vectors.”

A first nucleic-acid sequence is “operably” linked with a second nucleic-acid sequence when the first nucleic-acid sequence is placed in a functional relationship with the second nucleic-acid sequence. For instance, a promoter is operably linked to a coding sequence if the promoter affects the transcription or expression of the coding sequence. Generally, operably linked nucleic acid sequences are contiguous and, where necessary to join two protein coding regions, in reading frame.

Although methods and materials similar or equivalent to those described herein can be used in the practice or testing of the present invention, suitable methods and materials are described below. All publications, patent applications, patents and other references mentioned herein are incorporated by reference in their entirety. The particular embodiments discussed below are illustrative only and not intended to be limiting.

### BRIEF DESCRIPTION OF THE DRAWINGS

FIG. 1 is an alignment of the codon-optimized and wild-type (WT) luxA sequences.

FIG. 2 is an alignment of the codon-optimized and WT luxB sequences.

FIG. 3 is a graph showing average bioluminescence from stably transfected HEK293 cell lines (20 clones, tested for each clone type in triplicate). WTA/WTB=WT luxA and WT luxB. COA/WTB=codon-optimized luxA and WT luxB. COA/ICOB=codon-optimized luxA and codon-optimized luxB.

### DETAILED DESCRIPTION

The invention encompasses compositions and methods relating to codon-optimized nucleic acids encoding components of the bacterial luciferase system, a system that includes five individual genes (luxA, B, C, D, and E) that operate together to produce both the heterodimeric luciferase enzyme and its myristal-aldehyde substrate. The nucleic acids are codon-optimized by replacing one or more of the naturally occurring bacterial codons with codons that are optimized for expression in mammalian (e.g., human) cells. In the work described below, naturally occurring codons in the luxA and luxB genes from Photorhabdus luminescens were replaced with codons optimized for expression in mammalian cells. A similar strategy can be applied to obtain optimized sequences encoding other components of the bacterial luciferase system (e.g., luxCDE and frp nucleotide sequences). The codon-optimized bacterial luciferase enzyme system genes of the invention can be used to develop a mammalian cell bioluminescence bioreporter useful in various medical research and diagnostics applications.

The below described preferred embodiments illustrate various compositions and methods within the invention. Nonetheless, from the description of these embodiments, other aspects of the invention can be made and/or practiced based on the description provided below.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Various techniques using PCR are described, e.g., in Innis et al., PCR Protocols: A Guide to Methods and Applications, Academic Press: San Diego, 1990. PCR primer pairs can be derived from known sequences by known techniques such as using computer programs intended for that purpose (e.g., Primer, Version 0.5, ©1991, Whitehead Institute for Biomedical Research, Cambridge, Mass.). Methods for chemical synthesis of nucleic acids are discussed, for example, in Beaucage and Carruthers, Tetra. Letts. 22:1859-1862, 1981, and Matteucci et al., J. Am. Chem. Soc. 103:3185, 1981. Chemical synthesis of nucleic acids can be performed, for example, on commercial automated oligonucleotide synthesizers. Methods involving conventional biology and microbiology are also described herein. Such techniques are generally known in the art and are described in detail in methodology treatises such as Sambrook et al., supra; and Current Protocols in Molecular Biology, ed. Ausubel et al., John Wiley and Sons Publishing, New York, 2001 (with periodic updates).

### Nucleic Acids Encoding Lux Proteins

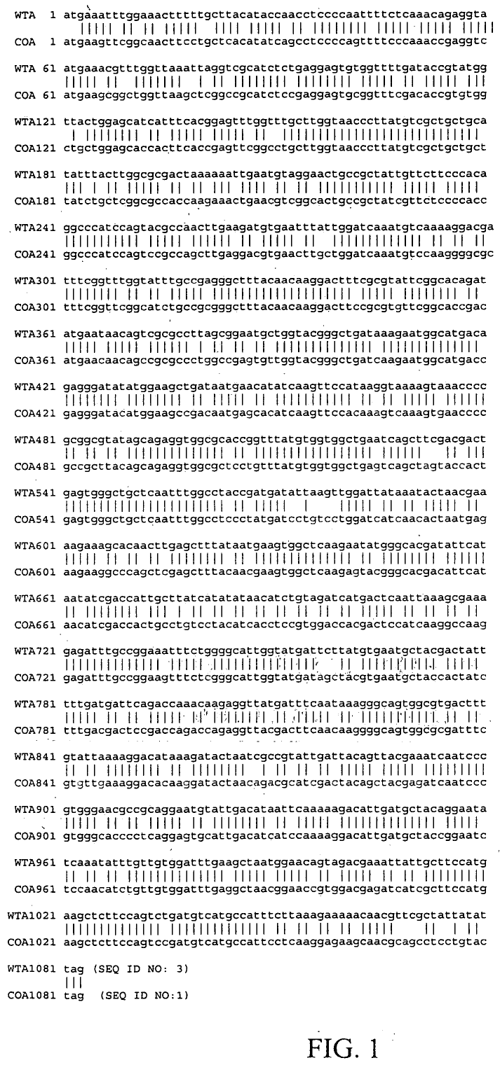

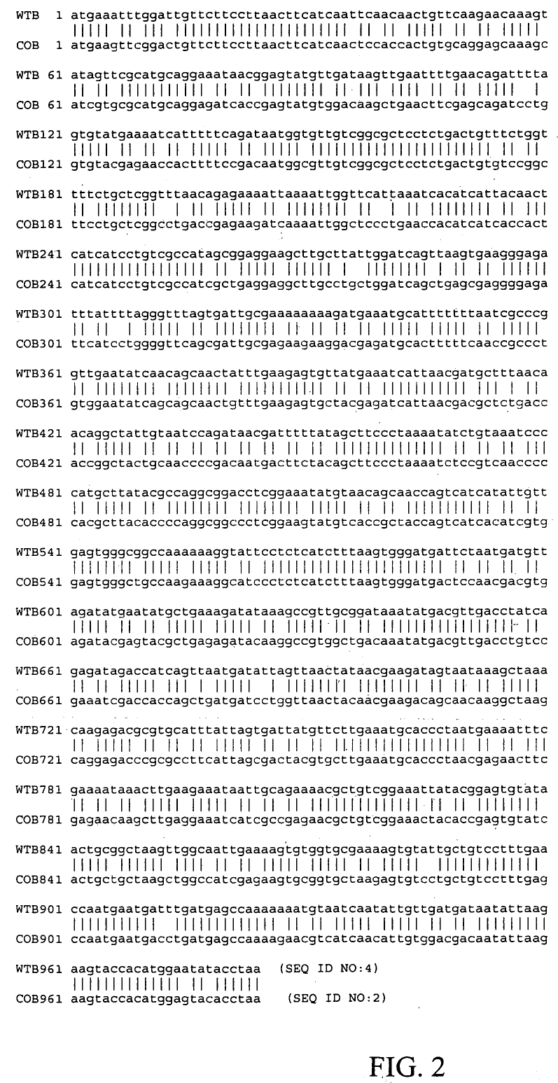

The invention provides nucleic acids encoding LuxA, LuxB, LuxC, LuxD, and LuxE proteins that have been modified for higher expression in mammalian cells. The sequence of a modified nucleic acid encoding luxA is listed herein as SEQ ID NO:1. A modified luxB gene sequence is listed herein as SEQ ID NO:2. As shown in the sequence alignments presented in FIGS. 1 and 2, the sequences of SEQ ID NO:1 and SEQ ID NO:2 have a number of codons differing from those in the native bacterial sequence. These codon substitutions facilitate higher expression of these lux genes in mammalian cells but do not change the amino acid sequence of the encoded protein. To further optimize expression of luxA and luxB in mammalian cells, potential splice sites and regulatory regions (e.g., transcriptional and translational stop sites) are absent from SEQ ID NOs: 1 and 2.

Nucleic acids encoding Lux proteins can be codon-optimized using any suitable technique. As an example, a nucleotide sequence optimized for expression in mammalian cells is determined by first analyzing the codons in the gene to be modified. These codons are then compared with codons commonly used in mammalian genes (See, e.g., Wada et al., Nucleic Acids Research 18(supplement):2367-2411, 1990) to identify where codon substitutions that facilitate increased expression in mammalian cells can be made. Once these sites have been determined, the starting nucleotide sequence is subjected to recombinant DNA technology to incorporate the desired codon substitutions. Techniques such as site-specific mutagenesis (Adelman et al., DNA 2:183, 1983) and PCR (Current Protocols in Molecular Biology supra, Chapter 8) can be used to incorporate nucleotide substitutions in a nucleotide sequence. In the examples described below, modified lux genes were constructed by a “recursive” PCR technique using synthesized oligonucleotides with overlapping ends as the template DNA (See, Prodromou and Pearl, Protein Engineering 5:827-829, 1992).

Nucleic acids of the invention may be in the form of RNA or in the form of DNA (e.g., cDNA, genomic DNA, and synthetic DNA). The DNA may be double-stranded or single-stranded. In the examples described herein, nucleic acids encoding LuxA, LuxB, LuxC, LuxD, and LuxE were derived from wild-type P. luminescens. Nucleic acid sequences which encode native P. luminescens LuxA, LuxB, LuxC, LuxD and LuxE proteins are listed in Genbank as accession numbers AF403784, M62917, M55977, M90092, and M90093, respectively. The amino acid sequences of native P. luminescens LuxA, LuxB, LuxC, LuxD, and LuxE proteins are listed in Genbank as accession numbers AAK98554, AAK98555, AAK98552, AAK98553, and AAK98556, respectively. Nucleic acids encoding LuxA, LuxB, LuxC, LuxD, and LuxE derived from other strains or organisms might be used so long as they can be expressed in mammalian cells to generate luminescence. For example, nucleic acids encoding LuxA, LuxB, LuxC, LuxD, and LuxE proteins from Vibrio harveyi, P. luminescens, Photobacterium phosphoreum, Photobacterium leiognathi, and Shewanella hanedai might be used in the invention. The Lux proteins from P. luminescens are preferred for mammalian expression applications because they are heat stable at 37° C. (Szittner and Meighen, J. Biol. Chem..265:16581-16587, 1990).

Other nucleic acid molecules within the invention are those that encode fragments, analogs and derivatives of LuxA, LuxB, LuxC, LuxD and LuxE proteins and those that encode mutant forms of these proteins or non-naturally occurring variant forms of these proteins. For example, nucleic acids that have a nucleotide sequence that differs from native luxA, luxB, luxC, luxD and luxE in one or more bases might be used. For instance, the nucleotide sequence of such variants can feature a deletion, addition, or substitution of one or more nucleotides of a native luxA, luxB, luxC, luxD or luxE.

frp Nucleic Acids and NAD(P)H-FMN Oxidoreductase/FMN Oxidoreductase Protein

In addition to the lux nucleic acids, nucleic acids encoding NAD(P)H-flavin oxidoreductase protein (FMN oxidoreductase, also known as NAD(P)H-FMN oxidoreductase) might also be optimized for expression in mammalian cells in order to enhance the luminescence generated using the Lux system. NAD(P)H-flavin oxidoreductases (flavin reductases (FR)) are a class of enzymes that catalyze the reduction of flavin by NAD(P)H. The complete luciferase enzyme is a flavin monooxygenase that binds a reduced flavin molecule (FMNH₂) as a specific substrate. (Lei et al., J. Bacteriol. 176:3552-3558, 1994). The supply of FMNH₂ in mammalian cells is limiting, and this limitation has been shown to hamper bioluminescence generation significantly. Adequate levels of FMNH₂ in a mammalian cell can be attained by exogenous expression of a flavin reductase enzyme (e.g., FMN oxidoreductase) or by simply adding th6eu enzyme to the system. Bioluminescence levels eukaryotic cells may therefore be increased by increasing expression of FMN oxidoreductase in the cells. This may be achieved by the same codon optimization process described above for Lux system components.

To examine Lux system-induced luminescence, FMN oxidoreductase from V. harveyi (See Genbank accession number AAA21331 and UO8996), V. fischeri, Escherichia coli, and Helicobacter pylori can be used. The protein itself may be added to the Lux system components (e.g., in a cell or cell lysate) or the protein can be expressed from nucleic acids, e.g., nucleic acids that have been codon-optimized for expression in mammalian cells by the process described above.

### Codon Substitutions

Table 1 shows the preferred codons for gene expression in mammalian cells. The codons at the left represent those most preferred for use in mammalian genes, with mammalian usage decreasing towards the right. A codon sequence is preferred for mammalian expression if it occurs to the left of a given codon sequence in the second column of Table 1. Optimally, but not necessarily, less preferred codons in a non-mammalian polynucleotide coding sequence are mammalianized by altering them to the codon most preferred for that amino acid in mammalian gene expression. In making general mammalianizing changes, codons to be mammalianized can be identified by those of skill in the art from studying the information presented herein in Table 1 and from codon usage information from other sources (Sharp et al., Gene 215:405-413, 1998; Sharp et al., J. Mol. Evol. 37:441-456, 1993; and Amicis and Marchetti, Nucl. Acids Res. 28:3339-3345, 2000). For example, in utilizing the information in Table 1, one would compare the frequency of the bacterial codon against the frequency of those codons commonly used in mammalian genes, and make any appropriate changes. By way of example only, consider the amino acid leucine; the codon TTA is used nine times in the luxA and luxB genes, but this codon corresponds to only the fifth preferred codon in mammalian genes. Changing the leucine codons would thus make an appropriate starting point for creating a mammalianized gene (i.e., one codon-optimized for expression in mammalian cells).

Further changes that can be made following an analysis of Table 1 are to change the arginine codons of CGT, which is the last choice for use in the mammalian genome, to a more preferable codon such as CGC or AGG; changing codons such as TCA to more preferred codons such as TCC and AGC; optimizing threonine codons to ACC; avoiding the use of the proline codon CCG; changing the alanine codon GCG to the most preferred mammalian codon GCC; avoiding the use of the predominant glycine codons GGA and GGT and replacing these with those preferred in mammalian genes, GGC and GGG; substituting the frequently occurring valine codon GTA with the codon GTG that is favored in the mammals; and avoiding the isoleucine codon ATA and substituting this with the preferred mammalian codon ATC. Given this information, it will be understood that when introducing changes into nucleic acids encoding components of the bacterial luciferase system, it is preferred to introduce a codon from the left of the second column in Table 1.

In the examples described below, codon-optimized luxA and luxB nucleic acids were created using a number of steps. First, codon usage frequencies in the sequences of WT luxA and luxB were compared to codon usage frequencies in the ten most highly expressed mammalian genes. The luxA and luxB sequences were then altered such that the codon usage frequencies matched the codon usage frequencies of the highly expressed mammalian genes. For example, if a particular codon was not used at all in the highly expressed mammalian genes, this codon was removed from the luxA and luxB sequences. As another example, if a particular codon was used at a frequency of 75% in the highly expressed mammalian genes, the WT luxA and luxB sequences were modified such that this particular codon is used at a frequency of 75%. After the appropriate codon substitutions were made, the codon-optimized luxA and luxB sequences were further modified. All potential splice sites were removed, all transcription and translation start and stop sites were removed, and a number of transcription factor binding sites were removed. Methods of incorporating codon substitutions are described in Zhang et al., Biochemical Society Transactions 30:952-958, 2002; Gruber and Wood, Abstracts for the International Symposium for ISBC, Monterey, Calif., 2000; and U.S. Pat. No. 5,968,750.

TABLE 1
Preferred DNA Codons For Mammalian Use

| | |
|---|---|
| Alanine | GCC, GCT, GCA, GCG |
| Cysteine | TGC, TGT |
| Aspartic acid | GAC, GAT |
| Glutamic acid | GAG, GAA |
| Phenylalanine | TTC, TTT |
| Glycine | GGC, GGG, GGA, GGT |
| Histidine | CAC, CAT |
| Isoleucine | ATC, ATT ATA |
| Lysine | AAG, AAA |
| Leucine | CTG, TTG, CTT, CTA, TTA |
| Methionine | ATG |
| Asparagine | AAC, AAT |
| Proline | CCC, CCT, CCA, CCG |
| Glutamine | CAG, CAA |
| Arginine | CGC, AGG, CGG, AGA, CGA, CGT |
| Serine | AGC, TCC, TCT, AGT, TCA, TCG |
| Threonine | ACC, ACA, ACT, ACG |
| Valine | GTG, GTC, GTT, GTA |
| Tryprophan | TGG |
| Tyrosine | TAC, TAT |

### Vectors/Regulatory Elements

In some applications of the invention, one or more of the codon-optimized nucleic acids encoding LuxA, LuxB, LuxC, LuxD, LuxE and FMN oxidoreductase are incorporated into a vector and/or operably linked to one or more regulatory elements. Any suitable vector that includes a replication system and sequences that are capable of transcription and translation of a polypeptide-encoding sequence in a given host cell may be used. Examples of suitable expression vectors include pIRES (Clontech, San Jose, Calif.) and variations thereof, and pcDNA (Invitrogen, Carlsbad, Calif.) and variations thereof. Expression vectors within the invention may include-regulatory elements that facilitate expression of a polypeptide in a host cell. For the practice of the present invention, conventional compositions and methods for preparing and using vectors and host cells are employed, as discussed, e.g., in Sambrook et al., supra, or Ausubel et al., supra.

Operably linked nucleic acid sequences can be contiguous and, where necessary to join two protein coding regions, in reading frame. Operably linked nucleic acid sequences can also be non-contiguous. Examples of regulatory elements include promoters, enhancers, initiation sites, polyadenylation (polyA) tails, internal ribosome entry site (IRES) elements, response elements, and termination signals.

To achieve appropriate levels of LuxA, LuxB, LuxC, LuxD, LuxE and FMN oxidoreductase proteins, any of a number of promoters suitable for use in the selected host cell may be employed. For example, constitutive promoters of different strengths can be used to express the LuxA, LuxB, LuxC, LuxD, LuxE and FMN oxidoreductase proteins. Inducible promoters may also be used in compositions and methods of the invention. To achieve regulated expression of LuxA, LuxB, LuxC, LuxD, LuxE and FMN oxidoreductase proteins in mammalian cells, a constitutive cytomegalovirus (CMV) promoter is preferred, however, any promoter known to function in mammalian cells may be used.

To increase levels of bacterial luciferase proteins, nucleic acids encoding these proteins are operably linked to any of a number of enhancers suitable for use in mammalian (e.g., human) cells. One example of an enhancer that may be useful is the SP163 site, an untranslated region in the mouse genome that has been shown to increase translation several-fold when placed upstream of genes in mammalian cells (Stein et al., Molecular and Cellular Biology 18:3112-3119, 1998.

To facilitate expression of a nucleic acid, the nucleic acid may be operatively linked to an IRES element. IRES elements allow ribosomes to bind directly at an AUG start codon rather than requiring initial recognition at the 5′ cap site and subsequent scanning for the start site (Hellen and Sarnow, Genes Dev. 15:1593-1612, 2001). If the AUG start site is located within the open reading frame, translation can be initiated internally and a monocistronic mRNA essentially becomes multiply-cistronic. The insertion of an IRES fragment between lux (e.g., luxA, luxB, luxC, luxD, luxE) nucleic acids facilitates bicistronic synthesis of Lux proteins. Similarly, insertion of an IRES fragment between lux (e.g., luxA, luxB, luxC, luxD, luxE) and frp nucleic acids facilitates bicistronic synthesis of Lux and FMN oxidoreductase proteins. In the examples below, the IRES of the encephalomyocarditis virus (EMCV) was used. See Meilke et al., Gene 254:1-8, 2000 and Harries et al., Journal of Gene Medicine 2:243-249, 2000.

### Cells Containing Lux System Nucleic Acids

The nucleic acids of the invention might be introduced into a cell as a reporter system, e.g., to measure the level of expression of a gene to which the system is linked. Although the codon-optimized nucleic acids of the invention are optimized for use in mammalian-cells, the nature of mammalian codon usage allows expression of the nucleic acids in non-mammalian cells such as those from organisms such as zebrafish, yeast (e.g., Candida species), and plants (e.g., tobacco, canola, arabidopsis).

Cells of the invention may include mammalianized nucleic acids encoding LuxA, LuxB, LuxC, LuxD, LuxE and FMN oxidoreductase proteins as episomes (e.g., plasmids) or as chromosomally integrated nucleic acids. In some applications integration of heterologous genes into the chromosome is preferred for long-term stability of gene expression. To integrate nucleic acids encoding LuxA, LuxB, LuxC, LuxD, LuxE and FMN oxidoreductase proteins into a mammalian cell chromosome, a number of known methods may be employed. See, Ryan and Sigmund Semin, Nephrol, 22:154-160, 2002; Harris et al., Anal. Biochem. 310:15-26, 2002; Osumi and Inoue Methods 24:35-42, 2001; Bode et al., Biol. Chem. 381:801-813, 2002, Sambrook et al., supra; and Wu et al., J. Virology 72:5919-5926, 1998.

### Methods of Introducing Codon-Optimized Nucleic Acids Into Cells

The codon-optimized nucleic acids of the invention can be introduced into cells using any suitable technique. In the experiments described below, mammalianized nucleic acids were introduced into cells using a lipofectamine-based transfection technique. However, several other suitable modes of delivery are known and include the following: microinjection (Wall, R. J. Cloning Stem Cells 15:348-364, 1995), electroporation (Preat, V., Ann. Pharm. Fr. 59:239-244, 2001), calcium phosphate transfection (Sambrook et al., supra), DEAE dextran transfection (Sambrook et al., supra), polylysine conjugates (Lollo et al., Methods Mol. Med. 69:1-13, 2002), receptor-mediated uptake systems (Molas et al., Curr. Gene Ther. 3:468-485, 2003), liposomes (Harashima et al., Crit. Rev. Ther. Drug Carrier Syst. 19:235-275, 2002), lipid-mediated delivery systems (Felgner et al., Ann. N.Y. Acad. Sci. 772:126-139, 1995; Lasic and Templeton, Adv. Drug Delivery Rev. 20:221-266, 1996), intra-cellular targeting ligands (Harashima et al., Eur. J. Pharma. Sci. 13:85-89, 2001; Qian et al., Pharmacol. Rev. 54:561-587, 2002), virus-like particles (Ou et al., J. Gen. Virol. 80:39-46, 1999), and viruses (Kay et al., Nature Medicine 7:33-40, 2001).

### Methods of Characterizing Increased Expression

The invention also provides methods of characterizing increased expression of a codon-optimized (e.g., mammalianized) nucleic acid encoding a component of the bacterial luciferase system (e.g., LuxA-E, FMN oxidoredtuctase). Methods for characterizing increased expression of a component of the bacterial luciferase system include measuring mRNA transcript levels, protein levels, and luminescence (i.e., bioluminesence) produced by a cell. Methods of detecting and quantitating nucleic acids (e.g., mRNA) and proteins are well known in the art. For example, techniques such as Northern blotting and reverse transcriptase PCR (RT-PCR) may be used to detect and quantitate mRNA encoding a component of the bacterial luciferase system. To detect and quantitate a protein of the bacterial luciferase system (e.g., LuxA-E, FMN oxidoreductase), techniques such as Western blotting and in vitro transcription/translation assays are useful within the invention.

Luminescence emitted by a cell containing a codon-optimized nucleic acid encoding a component of the bacterial luciferase system can be quantified by any suitable means, e.g., electronic, optical, or mechanical transducer. In some applications, the cells may be incorporated in a bioluminescent bioreporter integrated circuit (BBIC), a whole-cell integrated chemical sensor. Cells are maintained in close proximity to the integrated circuit of the BBIC. The IC portion of the BBIC detects and quantifies the luminescence and reports this data to (in some cases wirelessly) a central data collection location. The major components of the IC are the integrated photodetectors, the signal processing, and the wireless circuitry. These major components are described in Simpson et al., Trends in Biotechnology 16:332-338, 1998, and Bolton et al., Sensors and Actuators B. Chem. 85:179-185, 2002. Electronic integrated circuits and biosensor devices are described in U.S. patent application Ser. Nos. 09/949,015 and 09/910,360. CMOS microluminometers that may be used in the invention are described in Simpson et al., Sensors and Actuators B. Chem. 72:134-140, 2001; and Bolton et al., Sensors and Actuators B. Chem. 85:179-185, 2002.

### Applications for Codon-Optimized Nucleic Acids Encoding Luciferase System Components

Codon-optimized nucleic acids of the invention are useful for expressing bacterial luciferase system components (e.g., LuxA-E, FMN oxidoreductase) in mammalian cells. The combination of the luxA-E genes constitutes a reporter system for gene expression and has been used as a bioluminescent bioreporter in a number of applications. A bioluminescence bioreporter incorporating the codon-optimized bacterial luciferase system nucleic acids disclosed herein is useful in various medical research and diagnostics applications. In one embodiment of the present invention, the optimized lux genes can be used to develop a real-time blood glucose monitoring system for diabetic therapy.

Bioluminescent bioreporter technology is a fundamental sensing mechanism widely incorporated into whole-cell, systems for the detection of various targeted chemical and biological agents. The utilization of reporter proteins as a quantitative signal for blood glucose has already been established by Kennedy et al. (J. Biol. Chem. 274:13281-13291, 1999) using the firefly luciferase (luc) genes and using GFP. However, in both of these systems, external manipulations had to be performed before the bioluminescent signal could be generated. Consequently, neither system was capable of self-directed operation in a continuous, real-time format.

In this exemplary embodiment of the invention, codon-optimized bacterial luciferase genes are introduced into a mammalian cell line to produce a truly autonomous, real-time sensor for blood glucose. In this embodiment, the constitutive promoters of the expression vectors are replaced with glucose inducible promoters (e.g. human insulin-1 promoter or human GIP promoter) so that the reporter protein will be inducible in a glucose-dependent fashion. The codon-optimized luxAB genes are under the control of a glucose inducible promoter such as the human GIP promoter. The cell also expresses the luxCDE genes required for the bioluminescence reaction. Bioluminescence is generated when the luxAB genes are induced in the presence of glucose.

These glucose sensing engineered cells can further be incorporated into an implantable bioluminescent bioreporter integrated circuit (BBIC) by adhering to the OASIC (optical application specific integrated circuits) surface followed by envelopment of the entire device in an immunoisolating and light-tight membrane. The development of bioluminescent bioreporter integrated circuit has been described in U.S. Pat. No. 6,117,643. These miniaturized, low-power telemetry devices are capable of wireless data relay (Simpson et al., Trends in Biotechnology 19:317-323, 2001). Upon implantation into the diabetic patient, such devices could serve as continuous glucose monitors linked through remote communication networks to internal or external insulin pumps to automatically maintain normoglycemia.

The use of eukaryotic cells as glucose sensors will also have important implications in the diagnostic monitoring of various other medically targeted physiological analytes. For example, the bioluminescent mammalian bioreporter technology disclosed herein can be used to generate cells that are capable of sensing signatures for tumor progression and metastasis or infectious diseases. These cells could then be integrated into a bioluminescent bioreporter integrated circuit format for whole-body monitoring and early warning.

In addition to its use as a medical diagnostic tool, a remote sensing bioluminescent bioreporter integrated circuit designed specifically for glucose could also be applied in closed-loop control of cell culture processes for more efficient and reproducible cell and tissue growth, for on-line process control in agricultural industries, and as a sensor in metabolic engineering applications.

### EXAMPLES

The present invention is further illustrated by the following specific examples, which should not be construed as limiting the scope or content of the invention in any way.

### Example 1

### Expression of WT luxA and luxB in Mammalian Cells

The luxA and luxB genes from P. luminescens were fused via a PCR-based strategy, inserted into E. coli, and screened for light production. Since the luxC, D, and E genes were not present to provide the aldehyde substrate, the addition of n-decanal was used in these tests to generate bioluminescence.

E. coli clones exhibiting a bioluminescent phenotype were sequenced and used to construct a mammalian luxAB bioluminescent system via insertion into the mammalian expression vector pcDNA3.1 (chromosomal expression) or pREP9 (episomal expression) (Invitrogen, Carlsbad; Calif.). Both vectors contain either a CMV or RSV promoter for high constitutive expression and a neomycin resistance gene for selection. In vitro transcription and translation of the engineered luxAB fusion was performed to determine if there would be any significant problems with codon usage in a mammalian system. The transcripts were derived from a T7 promoter present directly upstream of the multi-cloning site of the pcDNA 3.1 vector. The transcripts were then translated by a rabbit reticulocyte lysate system with the, incorporation of [³⁵S]methionine to produced a radioactively labeled protein. The expected 80 kDa fusion protein was produced in the luxAB construct. However, expression levels were approximately 100-fold less than the firefly luciferase (luc) control protein, illustrating that codon usage was not optimal for expression in mammalian cells.

To determine if the LuxAB fusion protein was being expressed in vivo, insertions were generated within three mammalian cell lines, Hela, COS-7, and HEK293. All cells were transfected with circular (for episomal expression) or linear (for integration into the chromosome) plasmid DNA. Transfection was accomplished using a liposome mediated transfection reagent according to the manufacturer's protocol (Gibco BRL, Carlsbad, Calif.). Stable cell lines were selected from antibiotic resistant colonies.

To verify the presence of the plasmid harboring the luxAB fusion, PCR was performed on each stable isolate using lux-specific primers. From each pCR positive, stable cell line, total RNA was extracted and relative levels of expression of the luAB fusion message were determined using a ³²P-labeled probe for the luxA gene. The results indicated that while all stable clones had luxA mRNA levels greater than background, the message level varied. The highest levels were identified in the HEK293 cells that harbored the genes as an episomal plasmid.

Bioluminescence levels were determined for these cells following cell destruction and the addition of n-decanal and FMNH₂. The light emission varied between cell types and clones. The greatest amount of light was produced by the HEK293 clones. These engineered cells generated bioluminescent signals as much as 35 times over background. Therefore, the LuxAB fusion protein was capable of generating a measurable bioluminescent response. However, the light levels observed were not adequate to ensure a rapid and sensitive response to a target analyte such as glucose.

### Example 2

### Codon-Optimized luxA and luxB

### Materials and Methods

Cell Culture and Plasmid Maintenance: E. coli Cells were Routinely Grown in Luria Bertani (LB) (Fisher Scientific, Pittsburgh, Pa.) broth containing the appropriate antibiotic selection with continuous shaking (200 rpm) at 37° C. Kanamycin and Ampicillin were used at final concentrations of 50 μg/ml and 100 μg/ml, respectively.

All cell culture reagents and media were obtained from Sigma Aldrich, (St. Louis, Mo.) unless otherwise stated. Mammalian cells were grown in the appropriate complete growth media containing 10% heat-inactivated horse serum, 0.01 mM non-essential amino acids and 0.1 mM sodium pyruvate in a Dubelco's minimal essential media base (DMEM) (4655). Cells were routinely grown at 37° C. in a 5% CO₂ atmosphere to confluency and split every three to four days by trypsinization at a 1:4 ratio and transferred into fresh complete growth media. Appropriate concentrations of antibiotic were used to maintain constructs after transfection according to susceptibility kill curve analysis. Kill curves were completed for each lot of antibiotic. The range of typical concentrations used for the selection of HEK293 cell line clones was between 450 μg and 650 μg/ml.

Determining Codon-optimized Sequence of P. luminescens luxA and luxB Genes: To determine a codon-optimized sequence for P. luminescens luxA and luxB genes, the codon ratios within the WT genes were analyzed and compared to optimal codon usage patterns from highly expressed (top 10%) mammalian genes. The optimal codon ratios were determined by information tabulated in Genbank. The overall ratio for usage of each codon within the WT genes was altered to more closely match mammalian codon usage. In general, low frequency codons were used rarely or not at all and higher frequency codons were used more often. The codons were replaced within the WT sequences in a random fashion. The sequence was further analyzed for any potential splice sites or other regulatory regions using the NetGene2 algorithm for prediction of potential acceptor and donor splice sites. Any potential splice sites were removed. Transcription factor binding sites were also identified, however, these sequences were too numerous to successfully eliminate. After the final codon-optimized sequence was determined, it was compared to the WT sequence using the Genescan prediction algorithm to evaluate the potential expression of the new sequence versus the WT.

Synthesizing the Codon-optimized luxA and luxB Genes : Once the codon-optimized sequence had been determined, oligonucleotides for each gene were designed that covered the complete sequence. Each oligo was designed with an 18-23 base pair overlap on the 5′ and 3′ ends with its adjacent oligos. These overlapping regions were designed with Tm values of 53° C.-56° C. Once the oligos were designed they were synthesized by Sigma Genosys (Sigma, St. Louis, Mo.) and polyacrylamide gel (PAGE) purified to ensure full-length products. Each oligonucleotide was placed into a PCR reaction with the following conditions; internal oligos (0.25 pmol), the two outermost oligos (25 pmols), dNTP mixture (200 nm), 1× Pfu buffer, 1× Pfu Enhancer solution, MgCl₂ (concentration determined experimentally) and 1 U of Pfu DNA polymerase (Stratagene, La Jolla, Calif.).

All PCR reactions were performed in 0.2 ml thin walled PCR tubes using a PTC-225 DNA Engine (MJ Research, Waltham, Mass.). For gene synthesis the following program was used; (1) initial denaturation 95° C. for 5 min, (2) 30 cycles of 94° C. for 1 min, 50and 68° C. for 2 min followed by (3) final extension 68° C. for 10 min. Resultant PCR products were run on 1% agarose gels in 1× TBE. Unfortunately, there were no detectable products of the correct size. As an alternative strategy, four separate reactions were set up with four adjacent oligos in each reaction. The two innermost primers were added at a final concentration of 0.25 pmols and the two outermost oligos were used as both template and primers at a concentration of 25 pmols. Each piece was then amplified using the parameters outlined above with the exception of the extension step was reduced from 2 min to 45 sec. The resultant PCR products were then gel purified using the Geneclean gel extraction kit according to the manufacturer's instructions (Bio101, Carlsbad, Calif.). The extracted products were quantified using a Dyna Quant 200 fluorometer (Hoefer Pharmacia Biotech Incorporated, San Francisco, Calif.) and placed into a second PCR reaction at equal molar concentrations (0.25 pmols). The two outermost (5′ and 3′) oligos were used as primers at a final concentration of 25 pmols. After the second PCR reaction, the products of the correct size were again gel purified as previously described. Because Pfu polymerase produces blunt end products, 3′ A overhangs were added to allow for TA TOPO cloning of the products. To accomplish this, the gel-extracted product was mixed with DATP (200 nM) 1× amplitaq buffer and 1U of Taq polymerase (Amersham Pharmacia, San Francisco, Calif.) and placed at 72° C. for 20-30 min. Immediately following the addition of the A's, the product was TA TOPO cloned into the pCR4 TOPO cloning vector (Invitrogen Corporation, Carlsbad, Calif.). Resultant colonies were then checked for insert by an EcoRI restriction digest and sequenced to ensure their integrity.

Site Directed Mutagenesis: Although the oligos were successfully joined into a double stranded synthetic gene, several point mutations were determined by sequencing. A number of clones for each gene were completely sequenced in an attempt to identify a flawless clone without success. To correct these errors, site directed mutagenesis was performed. First, for the codon-optimized luxA gene, two separate clones were used as templates. Site directed mutagenesis primers were designed to introduce the necessary changes. The complete luxA sequence was amplified in two separate sections that overlapped between the bases where the necessary changes were required. Each segment was gel-purified and then linked back together by a second round of PCR as described for the original gene synthesis. Subsequently, 3′ A overhangs were added and the product TA TOPO cloned into pCR4. Upon sequencing, a construct with the correct sequence was identified and termed pPA2. Site directed mutagenesis was also performed on the codon-optimized luxB sequence using overlapping primers designed to introduce the proper changes. The complete luxB sequence was amplified in three segments from two separate clones and subsequently linked by PCR as previously described. A construct of the correct sequence was produced and termed pPB2.

Construction of a Bicistronic Expression Vector: To compare the expression of the codon-optimized luxA and luxB genes to the WT, the pIRES vector was used (Clontech Corporation, Palo Alto, Calif.). This expression vector contains two multi-cloning sites separated by an internal ribosomal entry site (IRES) from encephalomyocarditis virus (EMCV). The IRES element allows for the expression of two genes (one cloned into each multi-cloning site) from a single constitutive CMV promoter. For comparison purposes, a WT luxA and luxB (pWTA-I-WTB) construct, a codon-optimized luxA and WT luxB (pCOA-I-WTB) construct and a codon-optimized luxA and codon-optimized luxB (pCOA-I-COB) construct were generated.

pWTA-I-WTB: To create this construct, the luxA gene from P. luminescens was amplified from pPLluxCDABE plasmid that harbors the complete luxCDABE cassette and unique NotI restriction sites were introduced on both the 5′ and 3′ ends of the luxA gene. The resultant PCR product was TA TOPO cloned into pCR4 TOPO to generate pNotIluxA. The luxA gene was then cloned into the MCS(A) of pIRES via the unique NotI restriction sites to generate pWTAI. Once this construct was confirmed by sequencing, the plasmid was purified using the Wizard midi-prep plasmid purification kit according to the manufacturer's instructions (Promega Corporation, Madison, Wis.). The luxB gene was cleaved via a 5′:XbaI and 3′ Spel site from pCRluxB and cloned in to the XbaI site within the MCS(B) of pWTAI to generate pWTA-I-WTB.

pCOA-I-WTB: To generate this construct, the codon-optimized luxA gene (COA) was cleaved from pPA2 via unique NotI restriction sites and, cloned into the MCS(A) of the pIRES vector (Clontech Corporation, Palo Alto, Calif.) to generate pCOAI. Once this construct was confirmed by sequencing, the plasmid was purified using the Wizard midi-prep plasmid purification kit according to the manufacturer's instructions (Promega Corporation, Madison, Wis.). The WT luxB gene was cleaved via a 5′ XbaI and 3′ Spel site from pCRluxB and cloned into the XbaI site within the MCS(B) of pCOAI to generate pCOA-I-WTB.

pCOA-I-COB: To generate this construct, the codon-optimized luxB (COB) gene was cleaved from pPB2 via introduced 5′ and 3′ XbaI sites and cloned into the MCS(B) from pCOAI to create pCOA-I-COB.

Ligation Reactions: Plasmid vectors and inserts were digested (2-6 h) with the appropriate enzymes (Promega Corporation, Madison, Wis.). Linearized vectors were dephosphorylated using a calf intestine alkaline phosphatase enzyme according for the manufacturer's instructions (Promega Corporation, Madison, Wis.). Both vector and insert DNA were gel purified from 1% agarose gels using the Geneclean gel extraction kit (Bio101, Carlsbad, Calif.). The recovered DNA was then quantified using a Dyna Quant 200 fluorometer (Hoefer Pharmacia Biotech Incorporated, San Francisco, Calif.) and ligations were set up as 20 μl reactions using a 3:1 molar ratio of insert to vector DNA. The ligation reactions were then incubated at 17° C. overnight.

Electroporation: Electrocompetent cells were prepared as outlined by the manufacturer (BTX, San Diego, Calif.). Electroporations were performed using the BTX Electroporator 600 with the following conditions: 40 μl cells, 1-2 μl ligation mixture, a 2.5 kV pulse for 4.7 ms using a 2 mm gap cuvette. After the pulse, cells were immediately resuspended in 1 ml of sterile LB and allowed to recover for 1 h at 37° C. (200 rpm). Cells were then plated on selective media containing the appropriate antibiotic.

Selection of Bacterial Clones: Resistant colonies were picked after 24 h and expanded to patches on grid plates. To test for proper insert presence and orientation, rapid boil plasmid mini-preps (Promega Corporation, Madison, Wis.) were done followed by the digestion of the plasmid with the appropriate restriction enzyme mixture according to the manufacturer's instructions (Promega Corporation, Madison, Wis.). Products were run on 1% agarose gels to determine if the banding pattern indicated the insert presence and proper orientation. Upon identifying-correct clones, the plasmids were further purified using the Wizard midiprep plasmid purification system according to the manufacturer's protocol (Promega Corporation, Madison, Wis.) and sequenced.

Sequencing: All constructs were sequenced to ensure their integrity. Sequencing was done in the University of Tennessee Molecular Biology Service Facility using an Applied Biosystems 3100 Genetic Analyzer sequencer (Foster City, Calif.).

Transfection of Mammalian Cells: Transfection of mammalian cell lines was done in six well poly-D-lysine coated tissue culture plates (Fisher Scientific, Pittsburgh, Pa.). Cells were split from stock cultures and inoculated into each well at approximately 1×10⁵ cells per well in complete growth media. The plate was then placed at 37° C. in a 5% CO₂ atmosphere for 1-2 days until the cells became 80-90% confluent. The day of transfection, the media was refreshed. DNA for transfections was purified from 100 ml overnight E. coli cultures using the Wizard Purefection plasmid purification kit to remove endotoxins according to the manufacturer's instructions (Promega Corporation, Madison, Wis.). For chromosomal integration, the plasmid DNA was linearized before transfection to increase proper integration.

HEK293 Cells: Purified plasmid DNA (3.2 μg) was mixed into 200 μl of serum-free DMEM in a 1.5 ml tube. In a second tube, 8 μl of Lipofectamine 2000 reagent (Invitrogen Corporation, Carlsbad, Calif.) was added to 200 μl of serum free DMEM. The lipofectamine mixture was added to the DNA mixture within 5 min and incubated at room temperature for 20 min. The entire mixture (400 μl total) was added directly to the appropriate well on the plate and rocked back and forth to ensure adequate mixing. Twenty-four hours post transfection, the complexes were removed and the media was replaced with fresh complete growth media supplemented with the appropriate antibiotic for selection.

Selection of Mammalian Cell Clones: Twenty-four hours post transfection, selective media was added to all wells and refreshed every three to four days. Within two weeks all control wells were dead and the transfected cells were forming small colonies on the plate surface. Colonies were separated from the rest of the well by placing a sterile chamber around the cell mass and sealing it with silicon (Fisher Scientific, Pittsburgh, Pa.). The media could then be removed and each colony could be trypsinized and transferred to individual tissue culture flasks. To accomplish this, after washing with a phosphate buffered saline (PBS) solution, 200 μl of a 1× Trypsin-EDTA solution (Sigma Aldrich, St. Louis, Mo.) was added directly to the chamber and incubated at 37° C. for 3 to 5 min. The trypsin-EDTA solution was then replaced with complete growth media and the cells were transferred to a 25 cm² tissue culture flask for propagation. Each clone was given a number and expanded to individual cell lines. Each line was split and maintained as described earlier with the addition of selective media. Twenty cell lines were propagated in this manner for each plasmid tested.

Bioluminescence Assays from Mammalian Cells: To determine bioluminescence potential from each cell line clone, total proteins were extracted and in vitro enzyme (bioluminescence) assays performed. To extract the proteins, the cells were trypsinized from the plate or flask surface using standard protocols and resuspended into 2.0 ml Sarstedt tubes (Fisher Scientific, Pittsburgh, Pa.). The cells were then centrifuged down and washed two times in sterile PBS to remove any residual media (Sigma Aldrich, St. Louis, Mo.). Cell pellets were then resuspended into 1 ml 0.1M potassium phosphate buffer pH 7.8 and disrupted by three consecutive cycles of freeze (30 s liquid N2) thaw (5 min at 37° C.) extraction. After disruption, the cell debris was pelleted by spinning the samples at 14,000×g for 5 min and the supernatant was used in the bioluminescence assay. To determine light intensity, the protein extract was mixed with 0.1 mM NAD(P)H, 4 μM FMN, 0.2% (w/v) BSA, 0.002% (w/v) n-decanal. Bioluminescence was measured using the FB14 luminometer (Zylux Corporation, Pforzheim, Germany) at a 1 s integration and reported as relative light units (RLU). To determine if FMNH₂ was a limiting factor for the bioluminescence reaction, a flavin oxidoreductase enzyme (1 U) isolated from V. harveyi (Roche Scientific, Indianapolis, Ind.) was added to the bioluminescence assay and the light levels were measured again for comparison.

Bioluminescence signals were normalized between samples and cell lines by dividing the RLU measurement by the total protein and reporting the bioluminescence as RLU/mg total protein. Protein concentrations were determined using the Coomassie Plus protein assay according to the manufacture's instructions (Biorad, Hercules, Calif.).

In Vitro Transcription/Translation: To determine if the lux genes could be translated in vitro in rabbit reticulocyte lysate, pIRES vector harboring the WT luxA (WTA), and codon-optimized luxA (COA) were transcribed and translated. First, the plasmid DNA containing the genes was digested at a unique XbaI restriction site at the 3′ end of the gene within the vector. This digestion linearized the plasmid and allowed for the generation of run-off transcript from the vector derived T7 promoter. Each gene was transcribed via T7 polymerase using the RiboMax large-scale transcription system (Promega Corporation, Madison, Wis.). Three individual transcription reactions were set up along with a positive T7 control and a negative control containing no template DNA. Each reaction was set up according to the manufacturer's protocol and then incubated at 37° C. for 1 h. Transcripts were quantified by absorbance (260/280) measurements (Beckman Coulter, Fullerton, Calif.). Ten micrograms per ml of total RNA transcript was then added to 50 μL (total volume) rabbit reticulocyte lysate translation reactions. Each reaction was gently mixed on ice according to the manufacturer's protocol for S³⁵ labeled protein generation and then incubated at 30° C. for 90 min (Promega Corporation, Madison, Wis.). Once translation was complete, 15 μl of each reaction was loaded onto a 12% SDS-PAGE mini-gel and run at 30 mA for 1 h. The gel was removed and dried at 60° C. with vacuum pressure using a model 443 Slab Dryer (BioRad, Hercules, Calif.) onto 3MM filter paper (Fisher Scientific, Pittsburgh, Pa.). To visualize the generated proteins, the gel was placed onto an intensifier screen overnight and specific activity was measured using the STORM 840 phosphoanalyzer (Molecular Dynamics, Piscataway, N.J.).

Genomic DNA Isolation and Southern Blotting: Genomic DNA from each clone was accomplished using the Wizard genomic DNA extraction kit according to the manufacturer's protocols (Promega Corporation, Madison, Wis.). After isolation each preparation was quantified using a Dyna Quant 200 fluorometer (Hoefer Pharmacia Biotech Incorporated, San Francisco, Calif.). In two separate reaction tubes restriction digestions were set up with 2.5 kg of DNA each using a BamHI restriction enzyme according to the manufacturer's instructions (Promega Corporation, Madison, Wis.). Digestions were carried out in a 37° C. water bath for four hours. After digestion the products were loaded and run on a 1% agarose gel at 30V for 6 hours. The gel was then stained with ethidium bromide and photographed before the transfer. The gel was then soaked for 15 min in a depurination solution (250 mM HCl) and 30 min in a denaturation solution (0.5M NaOH and 1M NaCl), rinsed with dH2O and then neutralized two times for 15 min, in (0.5M Tris/1.5M NaCl) before a final equalization in 20×SSC. The DNA was then transferred to Biotrans™ nylon membrane (ICN, Irvine, Calif.) using the Turbo blotter apparatus accordinig to the manufacturer's instructions (Schleicher and Schuell, Keene, N.H.).

Double stranded DNA probes were generated complementary to a 300 bp portion of the codon-optimized and WT luxA genes using standard PCR protocols with the incorporation of a [³²P] labeled dCTP nucleotide. The probe was purified by column purification according to the manufacturer's instructions (Stratagene, La Jolla, Calif.). The specific activity of the each probe was measured by scintillation counting (Beckman Coulter, Fullerton, Calif.). Double stranded probes were boiled for 10 min to denature the DNA and directly added in equal amounts of specific activity to each blot.

The blot was incubated with the probe at 65° C. overnight. After probe hybridization, the blot was washed 4 times in 20×SSC to remove any unbound activity. The wash temperatures were determined experimentally to achieve optimal probe binding without excess background activity. The blot was air dried and then placed on a phosphorescence intensifier screen (Molecular Dynamics, Piscataway, N.J.). Specific activity was measured using the STORM 840 phosphoanalyzer and the data analyzed using the ImageQuant data analysis software package (Molecular Dynamics, Piscataway, N.J.).

RNA Isolation and Blotting: At passage six, post transfection, selected cell line clones were expanded to 75 cm² tissue culture flasks. When the cells became 80-95% confluent, they were trypsinized to remove the cells from the surface and transferred to 2.0 ml Sarstedt tubes (Fisher Scientific, Pittsburgh, Pa.). Cells were centrifuged down and washed two times in sterile PBS (Sigma Aldrich, St. Louis, Mo.). Total RNA was then isolated from the cells using the RNeasy kit (Quiagen, Valencia, Calif.) according to the manufacturer's instructions for isolation of total RNA from mammalian cells. To remove any contaminating DNA, the RNA was digested for 30 min with DNaseI (Promega Corporation, Madison, Wis.). To remove the DNaseI enzyme, the clean-up procedure from the RNeasy kit was used (Quiagen, Valencia, Calif.). Total RNA was then, quantified using the Beckman DU-640 spectrophotometer absorbance at 260/280 (Beckman Coulter, Fullerton, Calif.).

Northern Blotting: Ten micrograms of total RNA were loaded onto a 0.8% agarose formaldehyde gel and run at 100V for 2 hrs. The gel was then stained in an ethidium bromide solution and visualized. The RNA was then transferred to a Biotrans™ nylon membrane (ICN, Irvine, Calif.) using a semi-dry electroblot transfer apparatus according to the manufacturer's instructions (CBS Scientific, San Francisco, Calif.).

A 26 base pair oligonucleotide was designed to specifically hybridize to the codon-optimized and WT luxa sequences. This oligonucleotide was then 3′ end labeled with a γ [³²P] dATP by T4 polynucleotide kinase according to the manufacturer's protocol (Promega Corporation, Madison, Wis.). The oligonucleotide probe was then purified by column purification as outlined by the manufacturer (Stratagene, La Jolla, Calif.). The specific activity of the probe was measured by scintillation counting (Beckman Coulter, Fullerton; Calif.) and added directly to the blot.

Double stranded DNA probes were generated complementary to a 300 bp portion of the codon-optimized and WT luxA genes using standard PCR protocols with the incorporation of a [³²P] labeled dCTP nucleotide. The probe was purified by column purification according to the manufacturer's instructions (Stratagene, La Jolla, Calif.). The specific activity of each probe was measured by scintillation counting (Beckman Coulter, Fullerton, Calif.). Double stranded probes were boiled for 10 min to denature the DNA and directly added in equal amounts of specific activity to each blot.

The blot was incubated with the probe at 50° C. overnight. After probe hybridization, the blot was washed 4 times in 20×SSC to remove any unbound activity. The wash temperatures were determinined experimentally to achieve optimal probe binding without excess background activity. The blot was air dried and then placed on a phosphorescence intensifier screen (Molecular Dynamics, Piscataway, N.J.). Specific activity was measured using the STORM 840 phosphoanalyzer and the data analyzed using the ImageQuant data analysis software (Molecular Dynamics, Piscataway, N.J.).

Protein Isolation and Western Blotting: To extract the proteins, cells were trypsinized from a plate or flask surface and resuspended into 2.0 ml Sarstedt tubes (Fisher Scientific, Pittsburgh, Pa.). The cells were then centrifuged down and washed two times in sterile PBS to remove any residual media (Sigma Aldrich, St. Louis, Mo.). Cell pellets were resuspended into 1 ml 0.1M potassium phosphate buffer pH 7.8 and disrupted by three consecutive cycles of freeze (30 s liquid N₂) thaw (5 min at 37° C.) extraction. After disruption, the cell debris was pelleted by spinning the samples at 14,000×g for 5 min and the supernatant was used as total soluble protein for Western blot analysis.

Protein concentrations were determined using the Coomassie Plus protein assay according to the manufacturer's instructions (Pierce, Rockford, Ill.). Equal amounts (100-250 μg) of protein were loaded onto a 12% SDS-PAGE gel. Minigels were run at 30 mA for approximately 2 h and larger slab gels were run at 30 mA overnight. The proteins were then electroblot transferred to PDVF membrane (Biorad, Hercules, Calif.) using a semi-dry electroblotter according to the manufacturer's instructions (CBS Scientific Company, Incorporated, Del Mar, Calif.). Blots were then blocked overnight in 5% nonfat dry milk and hybridized with a polyclonal antibody raised against a 16 amino acid LuxA polypeptide or a 16 amino acid LuxB polypeptide (Genemed Synthesis, Incorporated, San Francisco, Calif.). Antibodies were diluted in T-TBS (Tris Buffered Saline+3% Tween 20) at a 1:500 dilution and applied to the membrane at room temperature for 5 h to overnight. The blot was then washed several times in T-TBS and incubated with a Goat Anti-Rabbit second antibody that has been conjugated to alkaline phosphatase. The blot was then developed according to the manufacturer's protocol (Biorad, Hercules, Calif.).

Statistics: Statistical analysis of the data presented here was conducted using either the JMP (SAS Institute, Incorporated, Pacific Grove, Calif.) or Microsoft Excel (Microsoft, Seattle, Wash.) statistical software packages. Graphs were made using Sigma Plot software (SPSS, SAS Institute, Incorporated, Pacific Grove, Calif.) or Microsoft Excel (Microsoft, Seattle, Wash.). All error bars on graphs indicate one standard deviation of the mean. Significant differences were determined using either t-test or 1 way ANOVA analysis at a level of α=0.05.

### Results

Determining a Codon-optimized Sequence of P. luminescens luxA and luxB for Expression in Mammalian Cells: The ratio of codons in the WT luxA and luxB nucleotide sequences was compared to codon usage patterns of highly expressed (top 10%) mammalian genes according to the Genbank sequence database. It was determined that the codon usage patterns between P. luminescens and mammalian genes were extremely different. Therefore, to create an optimized version of the lux genes, the codon ratios were altered to more closely follow codon usage patterns in mammals. Higher frequency codons were used more often while rare codons were eliminated from the sequence entirely. Changes were made within the nucleotide sequence in a random fashion. This codon-optimized sequence was further analyzed for potential regions that may act as target splice sites or other regulatory signals. The sequence was then modified until all potential splice sites and the more obvious regulatory sequences were removed. A comparison of the final codon-optimized and WT lux sequences are shown in FIGS. 1 and 2. Once the codon-optimized sequence was finalized it was tested using the GENSCAN online algorithm that predicts protein expression levels of gene sequences in mammalian cells by comparing the sequence to known highly expressed genes within the matrix specified. The results of this analysis were encouraging and a predicted a significant increase in expression on both transcriptional and translational levels. Further, although verification was not possible, GENSCAN predicted a cleavage of the first twenty amino acids of the WT LuxA protein when expressed in mammalian cells. This cleavage was eliminated in the codon-optimized sequence and a fill length product was predicted to form.

Construction of the Codon-optimized luxA and luxB Genes: To evaluate the potential impact of codon optimization on the expression of the bacterial luciferase genes in mammalian cells, codon-optimized versions of luxA and luxB genes were synthesized in vitro. To generate functional genes, single stranded oligonucleotides (80-106 bp) were designed that spanned the entire gene sequence with overlapping (18-23 bp) regions. Four oligonucleotides were placed into a single PCR reaction to amplify segments of the genes individually. The two outside oligonucleotides were used as both template and primers for the amplification reaction and the internal oligos as template. Resultant PCR products of the appropriate size were placed into a second PCR reaction and the fragments were then amplified to link the pieces together using the two outermost oligonucleotides as primers. Products of the correct size were again purified and TA TOPO cloned to generate pPA2 and pPB2. Complete sequence analysis was performed and revealed several introduced errors that were subsequently corrected by site directed mutagenesis.

In Vitro Transcription and Translation of the WT and Codon-optimized luxA: To quickly evaluate the translation efficiency in a mammalian cell system of the codon-optimized and WT luxA genes, in vitro transcription and translation analysis was performed. The pIRES expression vector contains a bacteriophage T7 promoter region upstream of the MCS (A). This promoter was used to generate runoff transcripts of the WT and codon-optimized luxA sequences. The transcript was then translated in vitro using rabbit a reticulocyte lysate system that incorporates a ³⁵S methoinine into the polypeptide sequence and allows for easy detection. The codon-optimized LuxA protein (COA) was determined to be produced by this system approximately twenty-fold over the WT LuxA protein.

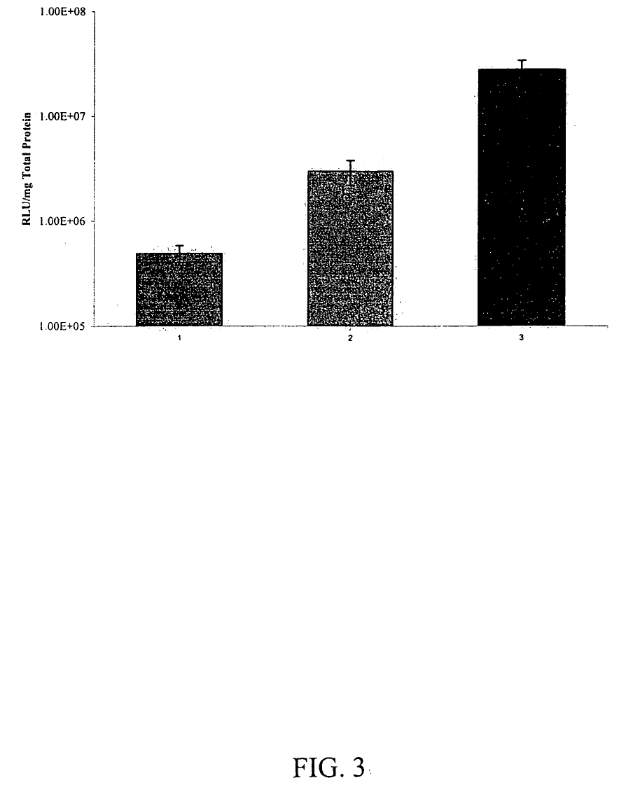

In Vivo Expression of the WT Versus Codon-optimized luxA and luxB Genes: To evaluate the optimized genes in vivo, WT and codon-optimized versions of the luxA and luxB genes were cloned into the pIRES mammalian expression vector to allow for bicistronic expression of both genes with only one selection marker. In one experiment, bioluminescence levels of 2.0×10⁷ RLU/mg total protein were achieved in stable HEK293 clones harboring a PA2-IRES-WTB construct. In another experiment, twenty stable clones (HEK293 cells) were selected for each construct along with one negative vector control. At passage three post transfection, each clone was tested in vitro for bioluminescence upon the addition of n-decanal and FMNH₂. These data revealed that each clonal cell line varied in its bioluminescence levels. The average bioluminescence from each gene combination is shown in FIG. 3. Based on these data, the two or three clones producing the highest bioluminescence levels were chosen for further study. At passage six, each clone selected was expanded into triplicate 75 cm² tissue culture flasks. From these cells, total genomic DNA, total RNA and soluble proteins were extracted for further analysis.

Determining Insertion Number in HEK293 Clones: To determine gene insertion number in each clone, a southern blot was performed using luxA probes generated to both the WT and codon-optimized luxA sequences. All of the cell lines tested had either one or two copies of the gene inserted with the exception of the COA/COB3 clone. To simplify further measurements, this clone was then disregarded for further bioluminescence comparisons.

Determination of luxA Message Levels in HEK293 Clones: To determine transcript levels, total RNA was extracted and Northern blot analysis was performed. The same probes that were used for Southern blot analysis were used in these experiments as well. Transcript levels were determined to be approximately equal with the exception of the WTA/WTB1 clone that had a lower amount of luxA transcript. The vector (NC) control had little to no background hybridization. The ethidium bromide stained 28S was included as an RNA loading reference.

Determination of LuxA Protein Levels in HEK293 Clones: Total soluble proteins from each clone were isolated by a series of freeze (liquid N₂) thaw (37° C.) cycles. Two hundred fifty micrograms of total protein were run on an SDS-PAGE gel and Western blot analysis was performed using a polyclonal LuxA antibody. LuxA protein was not detected in any of the WT luxA and luxB clones, only detected at very low levels in codon-optimized luxA with WT luxB clones, but readily detectable when both genes were codon-optimized. This increase in LuxA protein concentration was observed despite the fact that the levels of luxA mRNA transcript were relatively equivalent for all of the clones tested.

Bioluminescence Levels from WT Versus Codon-optimized Luciferase Genes: Bioluminescence levels were evaluated on whole cell extracts upon the addition of n-decanal and FMNH₂. Each clone was tested in triplicate from individual 35 cm² wells. Bioluminescence values were found to be greater than two orders of magnitude higher in cell lines harboring both a codon-optimized luxA and luxB (COA/COB) over that of the cell lines harboring the WT genes (WTA/WTB). The bioluminescence levels obtained increased in the order WTA/WTB<COA/WTB<COA/COB. Based on these data it was determined that codon optimization had a significant effect (p<0.05) on the bioluminescence potential from HEK293 cells.

### Example 3

### Expression of FMNH₂ in Mammalian Cells

### Materials and Methods

Cell Culture and Plasmid Maintenance was performed as described above in Example 2. The range of typical concentrations used for the selection of HEK293 cell line clones was between 450 μg/ml and 650 μg/ml of Neomycin G418 and 250 μg/ml and 400 μg/ml of Zeocin (Invitrogen Corporation, Carlsbad, Calif.).

Ligation reactions, electroporation, transfection of mammalian cells, and statistics for the experiments below are described in Example 2.

Construction of a Mammalian Expression System for frp: To generate a strain to overexpress the flavin oxidoreductase enzyme in mammalian cells, the frp gene was amplified from V. harveyi strain VHU08996 DNA. The gene was then TA TOPO cloned into the pCR4-TOPO cloning vector according to the manufacturer's instructions to generate pCR4frp (Invitrogen Corporation, Carlsbad, Calif.) and subsequently cut and ligated into the pcDNAHISMAX mammalian expression vector using introduced unique 5′BamHI and 3′ NotI restriction sites to generate pMaxfrp (Invitrogen Corporation, Carlsbad, Calif.). This expression vector possesses an SPC163 untranslated sequence upstream of the gene insert. This sequence has been shown to enhance translation between four- and five-fold over expression without the enhancer.

A second plasmid was generated to express the frp gene from V. harveyi by cloning the gene via the introduced unique 5′BamHI and 3′NotI restriction sites into the pcDNA3.1Zeo mammalian expression vector to generate pcfrpZeo.

Selection of Mammalian Cell Clones: Twenty-four hours post transfection, selective media was added to all wells and refreshed every three to four days. Because these transfections were conducted on a cell line that already harbored a Neomycin G418 resistance plasmid, the G418 was added at a concentration to maintain the plasmid and Zeocin was added to select for the second plasmid. When the COA/COB2 clone was co-transfected with the pMaxfrp, resistant clones never appeared within the transfected wells. Therefore, the pcfrp clone was generated to determine if the overexpression of the gene was causing a lethal product for the cells. Within two weeks after co-transfection with this plasmid construct, all control wells were dead and the transfected cells were forming small colonies on the plate surface. Colonies were separated from the rest of the well by placing a sterile chamber around the cell mass and sealing it with silicon (Fisher Scientific, Pittsburgh, Pa.). The media could then be removed and each colony could be trypsinized and transferred to individual. tissue culture flasks. To accomplish this, after washing with a PBS solution, 200 μl of a 1× Trypsin-EDTA solution (Sigma Aldrich, St. Louis, Mo.) was added directly to the chamber and incubated at 37° C. for 3 to 5 min. The trypsin-EDTA solution was then replaced with complete growth media and the cells were transferred to a 25 cm² tissue culture flask for propagation. Each clone was given a number and expanded to individual cell lines. Each line was split and maintained as described earlier with the addition of selective media. Nine cell lines were propagated in this manner.

In Vitro Bioluminescence Assays: To evaluate the bioluminescence potential from each cell line clone, total proteins were extracted and in vitro enzyme (bioluminescence) assays were performed. To extract the proteins, the cells were first trypsinized from the plate or flask surface and resuspended into 2.0 ml Sarstedt tubes (Fisher Scientific, Pittsburgh, Pa.). The cells were then centrifuged down and washed two times in sterile PBS to remove any residual media (Sigma Aldrich, St. Louis, Mo.). Cell pellets were then resuspended into 1 ml 0.1M potassium phosphate buffered pH 7.8 and disrupted by three consecutive cycles of freeze (30 s liquid N₂) thaw (5 min at 37° C.) extraction. After disruption, the cell debris was pelleted by centrifuging the samples at 14,000×g for 5 min and the supernatant was used in the bioluminescence assay. To determine light intensity, the protein extract was mixed with 0.1 mM NAD(P)H, 4 μM FMN, 0.2% (w/v) BSA, 0.002% (w/v) n-decanal. Bioluminescence was measured using the FB14 luminometer (Zylux Corporation, Pforzheim, Germany) at a 1 s integration and reported as relative light units (RLU). To evaluate the limitation of FMNH₂ for the bioluminescence reaction, a flavin oxidoreductase enzyme (1 U) isolated and purified from V. harveyi (Roche Scientific, Indianapolis, Ind.) was added to the mixture and light levels were measured again for comparison.

### Results

Evaluation of FMNH₂ Bioavailability in Mammalian Cells: To determine the overall bioavailability of the FMNH₂ substrate in mammalian cells, bioluminescence assays were performed and light measurements were taken before and after the addition of a purified flavin oxidoreductase enzyme. This enzyme in the presence of FMN and NAD(P)H reduces the FMN to the required FMNH₂ for the reaction. Bioluminescence levels from each of the cell line clones increased at least an order of magnitude after the addition of FMNH₂. These data illustrated that FMNH₂ was extremely limiting for the bioluminescence reaction from these engineered mammalian cell lines. However, in every case, the clones harboring the luxaA and luxB genes alone were able to produce bioluminescence levels above background without the addition of the flavin oxidoreductase enzyme indicating that some FMNH₂ was available within the cells for the reaction.

Expression of the Flavin Oxidoreductase Enzyme: In an attempt to overcome this limitation, the COA/COB2 clone (brightest clone) was co-transfected with an frp gene that was isolated from V. harveyi and cloned into a mammalian expression vector containing a translational enhancer region upstream of the multi-cloning site. When COA/COB2 clones were co-transfected with this plasmid, the HEK293 cells were not able to survive and as a result no clones were obtained from this construct. As an alternative approach, the frp gene was cloned into and expressed constitutively from the pcDNA3.1Zeo vector that allows for high constitutive expression but does not contain the SPC163 enhancer region. Nine stable cell line clones were obtained by resistance to toxic concentrations of both Neomycin G418 and Zeocin antibiotics simultaneously. Resultant clones were expanded to individual cell lines and tested for bioluminescence potential.

In Vitro Bioluminescence Assays: From in vitro bioluminescence assays (total protein extracts), the overall light levels increased with the expression of the frp gene at least an order of magnitude in both the absence and after the addition of exogenous flavin oxidoreductase versus the COA/COB2 clone without the frp gene tested under the same conditions. These data indicated that the expression of the frp gene was successful in producing an excess of available FMNH₂ within HEK293 cells. The further increase in bioluminescence after the exogenous addition of the purified oxidoreductase enzyme however, indicates that the system has yet to reach saturation. The bioluminescence levels obtained from the cell extract, in vitro, assays remained stable for several minutes before gradually declining to background levels. The light intensity could be increased back to peak levels upon exogenous addition of additional NAD(P)H to provide the reducing power for the flavin oxidoreductase enzyme and generate more FMNH₂. Thus the luciferase complex itself remained stable throughout the assay and bioluminescence levels were correlated to availability or decay of reduced FMN.

Whole Cell Bioluminescence Assays: Whole cell bioluminescence assays were performed to determine if these cell lines could produce adequate bioluminescence levels for use in gene expression analysis, much in the same way that firefly luciferase (Luc) is currently used today in several reporter applications. Average bioluminescence levels from the COA/COB2 clone were obtained that were at least two orders of magnitude greater than background levels (4×10⁴ RLU/s versus 380 RLU/s). The bioluminescence was further increased at least another order of magnitude when the frp gene was co-expressed along with the luciferase genes. All clones co-transfected to express the frp gene produced significantly more light than without the enzyme being expressed (p<0.05). Furthermore, there were significant differences between the nine frp clones as well. However, unlike the relatively stable nature of the bioluminescence signal from in vitro bioluminescence assays, the light levels from these whole cell clones resulted in a flash bioluminescent response. The maximum light output was obtained within 1 s of n-decanal addition and returned to background levels within five seconds. These levels could not be induced with the further addition of n-decanal or FMNH₂ to achieve a second peak in bioluminescent activity.

Stability of Bacterial Luciferase in Mammalian Cells Over Long Periods of Time: The stability of mammalian cell lines engineered to stably express the bacterial luciferase genes was monitored by performing bioluminescence assays over time. The bioluminescence levels remained relatively constant for forty passages, for every clone except WTA/WTB2 where the level radically deteriorated after passage thirty. Although the light levels for the other clones remained relatively stable during this time, other phenotypic changes occurred within the cells, including a lower binding affinity to the flask surface.

### Example 4

### Growth of Cells on Chip Surfaces

The bioluminescent HEK293 cells described above were easily grown and maintained on a chip surface. Amino-modified and PEG-modified chip surfaces were tested. Chips were placed in six well plates and cells in normal growth media were added to the chips. After a few hours, the cells adhered to the chip surface. Highly vital adhering cells were grown on amino-modified chip surfaces and proliferated for several days. Stagnant cells were grown on PEG-modifed chip surfaces. Cells were also placed on chip surfaces that had been treated with a growth-preventing compound in a particular pattern. The cells grew only where the compound was not present. The growth of cells on a chip surface is described in U.S. Pat. No. 6,117,643. Use of the Lux system as a reporter system is described in Sayler et al., Current Opinions In Biotechnology, 12:455-460, 2001; Vollmer et al., Applied and Environmental Microbiology, 63:2566-2571, 1997; Wallace et al., Microbial Ecology, 27:224-240, 1994; Prest et al., Letters of Applied Microbiology 24:355-360, 1997; Cai et al., Biodegradation 8:105-111, 1997; Tibazarwa et al., Journal of Bacteriology, 182: 1399-1409, 2000; Corbiser et al., Environmental Toxicology and Water Quality 11:171-177, 1996; Hay et al., Applied and Environmental Microbiology, 66:4589-4594, 2000; Khang et al., Journal of Microbiology and Biotechnology, 7:352-355, 1997; Contag et al., Molec. Microbiology 18:593-603, 1995; Francis et al., Infection and Immunity, 68:3594-3600, 2000; and Francis et al., Infection and Immunity, 69:3350-3358, 2001.

### Other Embodiments

This description has been by way of example of how the compositions and methods of the invention can be made and carried out. Various details may be modified in arriving at the other detailed embodiments, and many of these embodiments will come within the scope of the invention. For example, mammalianized Lux nucleic acids may be expressed in eukaryotic cells other than mammalian cells such as yeast cells. Therefore, to apprise the public of the scope of the invention and the embodiments covered by the invention, the following claims are made.

What is claimed is:

1. A nucleic acid comprising a codon-optimized nucleotide sequence encoding a component of a bacterial luciferase system.

2. The nucleic acid of claim 1, wherein the codon-optimized nucleotide sequence differs from a wild type nucleotide sequence that encodes the component of a bacterial luciferase system by at least one codon substitution selected from the group consisting of: TTT to TTC; TTA, CTA, TTG, and CTT to CTG or CTC; ATT and ATA to ATC; GTT and GTA to GTG or GTC; TCT, TCA, and TCG to TCC; CCA and CCG to CCC or CCT; ACT, ACA and ACG to ACC; GCA and GCG to GCT or GCC; TAT to TAC; CAT to CAC; CAA to CAG; AAT to AAC; AAA to AAG; GAT to GAC; GAA to GAG; TGT to TGC; CGT and CGA to CGC, CGG, and AGA; AGT to AGC; and GGT and GGA to GGC or GGG.

3. The nucleic acid of claim 1, wherein the component of a bacterial luciferase system comprises a LuxA polypeptide.

4. The nucleic acid of claim 3, wherein the codon-optimized nucleotide sequence is SEQ ID NO:1.

5. The nucleic of claim 1, wherein the component of a bacterial luciferase system comprises a LuxB polypeptide.

6. The nucleic acid of claim 5, wherein the codon-optimized nucleotide sequence is SEQ ID NO:2.

7. The nucleic acid of claim 1, further comprising a regulatory element operably linked to the codon-optimized nucleotide sequence.

8. The nucleic acid of claim 7, wherein the regulatory element comprises an enhancer.

9. A cell comprising a nucleic acid comprising a codon-optimized nucleotide sequence encoding a component of a bacterial luciferase system.

10. The cell of claim 9; wherein the cell is a mammalian cell.

11. The cell of claim 9, wherein the cell is immobilized on a substrate.

12. The cell of claim 9, wherein the codon-optimized nucleotide sequence differs from a wild type nucleotide sequence that encodes the component of a bacterial luciferase system by at least one codon substitution selected from the group consisting of: TTT to TTC; TTA, CTA, TTG, and CTT to CTG or CTC; ATT and ATA to ATC; GTT and GTA to GTG or GTC; TCT, TCA, and TCG to TCC; CCA and CCG to CCC or CCT; ACT, ACA and ACG to ACC; GCA and GCG to GCT or GCC; TAT to TAC; CAT to CAC; CAA to CAG; AAT to AAC; AAA to AAG; GAT to GAC; GAA to GAG; TGT to TGC; CGT and CGA to CGC, CGG, and AGA; AGT to AGC; and GGT and GGA to GGC or GGG.

13. The cell of claim 9, wherein the component of a bacterial luciferase system comprises a LuxA polypeptide.

14. The cell of claim 13, wherein the codon-optimized nucleotide sequence is SEQ ID NO:1.

15. The cell of claim 9, wherein the component of a bacterial luciferase system comprises a LuxB polypeptide.

16. The cell of claim 15, wherein the codon-optimized nucleotide sequence is SEQ ID NO:2.

17. The cell of claim 9, wherein the codon-optimized nucleotide sequence is operably linked to a regulatory element.

18. The cell of claim 17, wherein the regulatory element comprises an enhancer.

19. A method comprising the step of introducing into a mammalian cell a nucleic acid comprising a codon-optimized nucleotide-sequence encoding a component of a bacterial luciferase system.

20. The method of claim 19, wherein the codon-optimized nucleotide sequence differs from a wild type nucleotide sequence that encodes the component of a bacterial luciferase system by at least one codon substitution selected from the group consisting of: TTT to TTC; TTA, CTA, TTG, and CTT to CTG or CTC; ATT and ATA to ATC; QTT and GTA to GTG or GTC; TCT, TCA, and TCG to TCC; CCA and CCG to CCC or CCT; ACT, ACA and ACG to ACC; GCA and GCG to GCT or GCC; TAT to TAC; CAT to CAC; CAA to CAG; AAT to AAC; AAA to AAG; GAT to GAC; GAA to GAG; TGT to TGC; CGT and CGA to CGC, CGG, and AGA; AGT to AGC; and GGT and GGA to GGC or GGG.

21. The method of claim 19, wherein the component of a bacterial luciferase system comprises a LuxA polypeptide.

22. The method of claim 21, wherein the codon-optimized nucleotide sequence is SEQ ID NO:1.

23. The method of claim 19, wherein the component of a bacterial luciferase system comprises a LuxB polypeptide.

24. The method of claim 23, wherein the codon-optimized nucleotide sequence is SEQ ID NO:2.

25. The method of claim 19, wherein the codon-optimized nucleotide sequence is operably linked to a regulatory element.

26. The method of claim 25, wherein the regulatory element comprises an enhancer.
